(19) United States
(12) Patent Application Publication
(10) Pub. No.: US 2002/0072720 A1
(43) Pub. Date: Jun. 13, 2002

(54) Rigid soluble materials for use with needle-less infusion sets, sensor sets and injection devices and methods of making the same

(76) Inventors: Clifford W. Hague, Sherman Oaks, CA (US); Leif Bowman, Burbank, CA (US); Jason H. Safabash, Los Angeles, CA (US); John H. Livingston, Newport beach, CA (US); Adrian Gut, Los Angeles, CA (US); Neal Smith, Los Angeles, CA (US); David J. Vachon, Granada Hills, CA (US); Paul H. Kovelman, Simi Valley, CA (US)

Correspondence Address: MEDTRONIC MINIMED INC., 18000 DEVONSHIRE STREET, NORTHRIDGE, CA, 91325-1219, US

(21) Appl. No.: 09/736,666

(22) Filed: Dec. 11, 2000

(51) Int. Cl. A61M 5/00; A61M 25/00

(52) U.S. Cl. 604/264

(57) ABSTRACT

An insertion set for insertion into a skin of a user. The insertion set includes an insertable substantially insoluble flexible portion and a soluble material. The insertable substantially insoluble flexible portion is capable of remaining in the skin after insertion. The soluble material is coupled to the insertable insoluble flexible portion and facilitates piercing the skin, and the soluble material dissolves in the skin of the user. Also, the soluble material holds the substantially insoluble flexible portion in a rigid state. Preferably, the insertion set is an infusion set or a sensor set. The sets may include a cannula as part of the insertable substantially insoluble portion. The cannula may be formed from a flexible material or a flexible metal tube. Preferably, the soluble material is formed from a at least one saccharide, such as a monosaccharide or a polysaccharide, a protein, a starch, other biocompatible materials, or the like.

### FIELD OF THE INVENTION

This invention relates to insertion sets, such as infusion sets and sensor sets, and, in particular embodiments, to infusion sets, sensor sets and injection devices that are needle-less and include a soluble material on a cannula and/or sensor.

### BACKGROUND OF THE INVENTION

Over the years, infusion sets have been used to infuse fluids from external infusion devices, such as those as generally described in U.S. Pat. Nos. 4,562,751; 4,678,408; and 4,685,903, which are herein incorporated by reference. Early infusion sets used a hard metal needle connected to the end of a long, flexible tube connected to the infusion device. The metal needle was inserted under the skin or into a vein to deliver the fluid to the infusion site. The tube and needle were often taped in place to inhibit accidental removal. Although generally simple in construction, this type of infusion device suffered from several drawbacks. For instance, the needle is sharp and can continually irritate the insertion site as it is jarred. In addition, the needle is stiff and resists lateral movement as the skin is flexed. Finally, when the set is removed, there is a sharp needle that could inflict accidental needle sticks and must be properly disposed of in a sharps container, or the like.

To overcome some of these drawbacks, infusion sets that utilize a soft cannula have been developed. For instance, a typical soft cannula infusion set is disclosed in U.S. Pat. No. 4,755,173 issued Jul. 5, 1988 to Konopka et al., which is herein incorporated by reference. A soft cannula does not generally continue to irritate the insertion site and tends to move flexibly with the skin. Thus, infusion sets with soft cannulas are often more comfortable to wear. However, due to the nature of a soft cannula, an insertion needle is used to place the cannula under the skin. Therefore, although more comfortable to wear, there is still a needle present that must be properly disposed of as described above. In addition, the use of an insertion needle tends to complicate the structure of the infusion set to accommodate the insertion needle, tending to make these sets more difficult to manufacture than a simple needle infusion set.

Sensor sets often utilize a soft thin film flexible sensor that is contained inside of a hollow needle during insertion of the sensor, and these suffer from similar drawbacks associated with the soft cannula infusion sets described above. A typical sensor set is disclosed in U.S. Pat. No. 5,954,643 issued Sep. 21, 1999 to Van Antwerp et al., which is herein incorporated by reference.

### SUMMARY OF THE DISCLOSURE

It is an object of an embodiment of the present invention to provide an improved infusion and/or sensor set, which obviates for practical purposes, the above mentioned limitations.

Embodiments of the present invention are directed to an insertion set for insertion into a skin of a user, the insertion set includes an insertable substantially insoluble flexible portion and a soluble material. The insertable substantially insoluble flexible portion is capable of remaining in the skin after insertion. The soluble material is coupled to the insertable substantially insoluble flexible portion and facilitates piercing the skin, and the soluble material dissolves in the skin of the user. Also, in other embodiments, the soluble material holds the substantially insoluble flexible portion in a rigid state. In preferred embodiments, the insertion set is an infusion set or a sensor set. In particular embodiments, the insertable substantially insoluble portion is a flexible cannula. In further embodiments, the cannula is formed from a flexible plastic material or a flexible metal tube. Preferably, the soluble material is formed from at least one saccharide. In particular embodiments, the at least one saccharide is a monosaccharide or a polysaccharide. In other embodiments, the soluble material is formed from a starch, a protein, soluble biocompatible material, or the like.

In preferred embodiments, the insertion set is adapted to be inserted through the skin and/or placed into subcutaneous tissue. In particular embodiments, the insertion set is an infusion set that includes an at-site disconnect for use with infusion tubing. In other embodiments, the insertion set is an infusion set that includes a side disconnect for use with infusion tubing. In still other preferred embodiments, the insertable substantially insoluble flexible portion is a sensor. In particular embodiments, the insertable substantially insoluble flexible portion further includes a cannula surrounding the sensor. In still other embodiments, the insertable substantially insoluble flexible portion is porous. In further embodiments, the insertable substantially insoluble flexible portion is a cannula with at least one side port.

In preferred embodiments, the soluble material dissolves in the skin in under ten minutes. In other embodiments, the soluble material dissolves in the skin in under 1 hour. In particular embodiments, the soluble material includes at least one flange to improve structural strength. In further embodiments, the soluble material is formed from multiple layers of materials with different properties.

In another embodiment of the present invention, an infusion device for insertion into a skin of a user to infuse a fluid includes an insertable substantially insoluble flexible portion and a soluble material. The insertable substantially insoluble flexible portion is capable of remaining in the skin after insertion to deliver a fluid, and the soluble material is coupled to the insertable substantially insoluble flexible portion that facilitates piercing the skin, and the soluble material dissolves in the skin of the user. In still another embodiment of the present invention, a syringe device for insertion into a skin of a user to deliver an injection includes an insertable substantially insoluble flexible portion and a soluble material. The insertable substantially insoluble flexible portion capable of delivering an injection to the skin of the user after insertion, and the soluble material is coupled to the insertable substantially insoluble flexible portion that facilitates piercing the skin, and the soluble material dissolves in the skin of the user.

Other features and advantages of the invention will become apparent from the following detailed description, taken in conjunction with the accompanying drawings which illustrate, by way of example, various features of embodiments of the invention.

### BRIEF DESCRIPTION OF THE DRAWINGS

A detailed description of embodiments of the invention will be made with reference to the accompanying drawings, wherein like numerals designate corresponding parts in the several figures.

FIG. 1 is a cross-sectional view illustrating an insertion set in accordance with a first embodiment of the present invention;

FIG. 2 is a cross-sectional view of an insertion set in accordance with a second embodiment of the present invention;

FIG. 3 is a cross-sectional view of a cannula of the insertion set as shown along the line 3-3 of FIG. 1;

FIG. 4 is a cross-sectional view of a cannula of the insertion set in accordance with a third embodiment of the present invention;

FIG. 5 is a cross-sectional view of the cannula of the insertion set as shown along the line 5-5 in FIG. 4.

FIG. 6 is a cross-section of a cannula for use with an insertion set in accordance with a fourth embodiment of the present invention.

FIG. 7 is a cross-section of a cannula for use with an insertion set in accordance with a fifth embodiment of the present invention.

FIG. 8 is a cross-section of a cannula for use with an insertion set in accordance with a sixth embodiment of the present invention.

FIG. 9 is a cross-section of an infusion set with a cannula in accordance with a seventh embodiment of the present invention.

FIG. 10 is a cross-section of an infusion set that utilizes a hard cannula in accordance with an eighth embodiment of the present invention.

FIG. 11 is a cross-sectional diagram of an alternative cannula for use in an insertion set in accordance with the embodiment of the present invention shown in FIG. 7.

FIG. 12 is a perspective view of a sensor set in accordance with a ninth embodiment of the present invention.

FIG. 13 is a partial cross-section of the sensor set as shown along the line 13-13 in FIG. 12.

FIG. 14 is a top perspective view of an infusion set in accordance with a tenth embodiment of the present invention.

FIG. 15 is a side perspective view of the infusion set of FIG. 14 during insertion into skin (or tissue).

FIG. 16 is a side perspective view of the infusion set of FIG. 14 after insertion and placement in the skin (or tissue).

FIG. 17 is a cross-sectional view of an infusion device in accordance with an eleventh embodiment of the present invention.

FIG. 18 is a cross-sectional view of a syringe device in accordance with a twelfth embodiment of present invention.

FIG. 19 is a partial cross-sectional view of an insertion device and element in accordance with a thirteenth embodiment of the present invention.

### DETAILED DESCRIPTION OF THE PREFERRED EMBODIMENTS

As shown in the drawings for purposes of illustration, the invention is embodied in an insertion set with a needle-less cannula or sensor. In preferred embodiments of the present invention, the infusion sets are for the infusion of fluids, such as insulin or the like, into subcutaneous tissue. However, it will be recognized that further embodiments of the invention may be used to infuse other fluids, such as saline, medication, drugs, vitamins, hormones or the like, and may be placed into other types tissue (hereinafter “skin), such as skin, dermal, sub-dermal, cutaneous, subcutaneous, or the like, and/or may be used in animal skin. Further embodiments are directed to a sensor set to determine the level of an analyte, such as glucose or the like, in the subcutaneous tissue. However, other embodiments may be used to determine the levels of other analytes or agents, characteristics or compositions, such as hormones, cholesterol, medication concentrations, viral loads (e.g., HIV), or the like. In still further embodiments, the sensor set may be placed in contact with other types of tissue, such as muscle, lymph, organ tissue, veins, arteries or the like, and used in animal tissue. Embodiments of the sensor set may be used to record sensor readings on an intermittent or continuous basis.

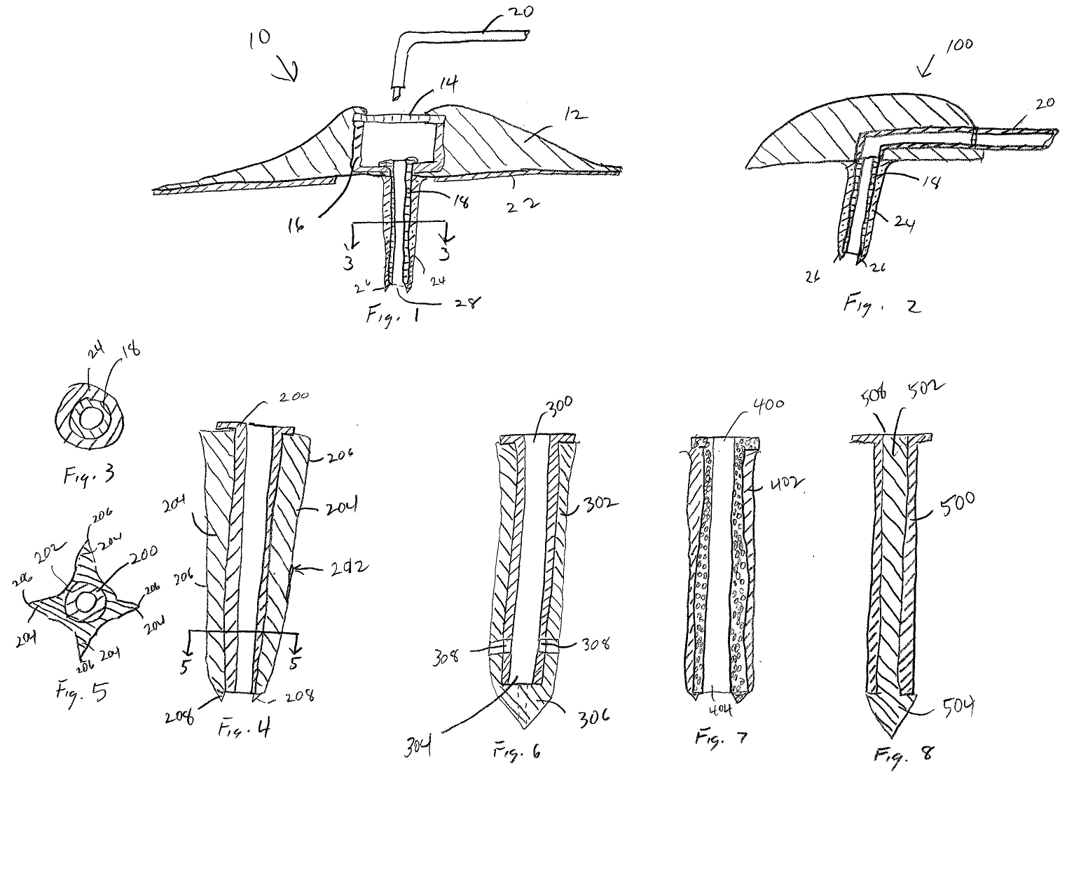

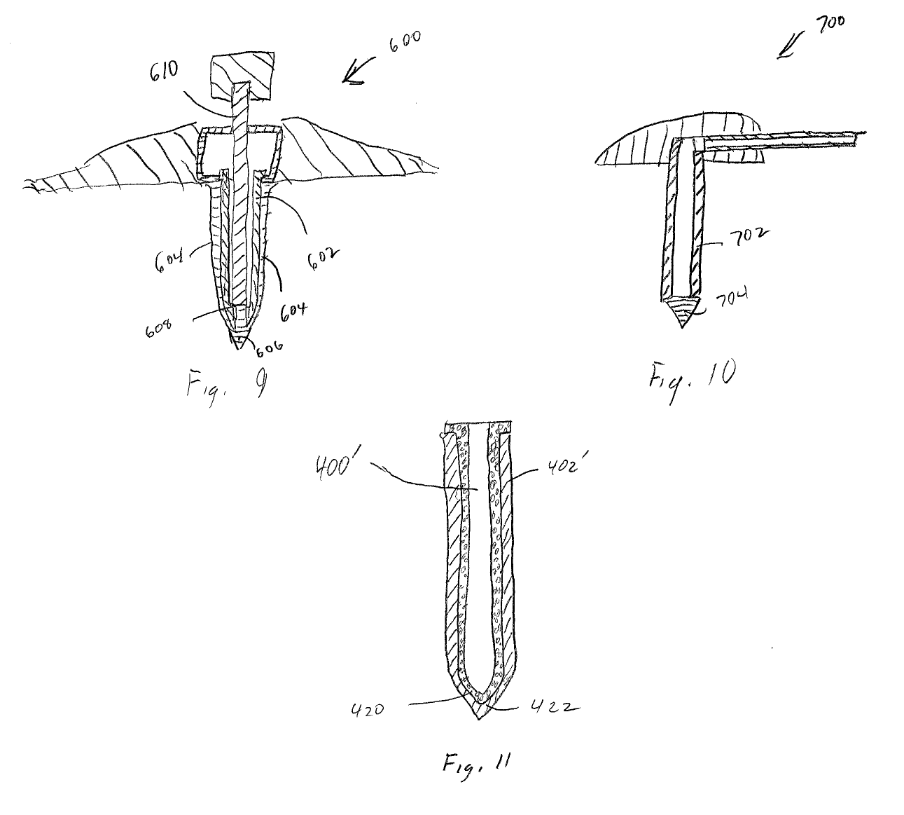

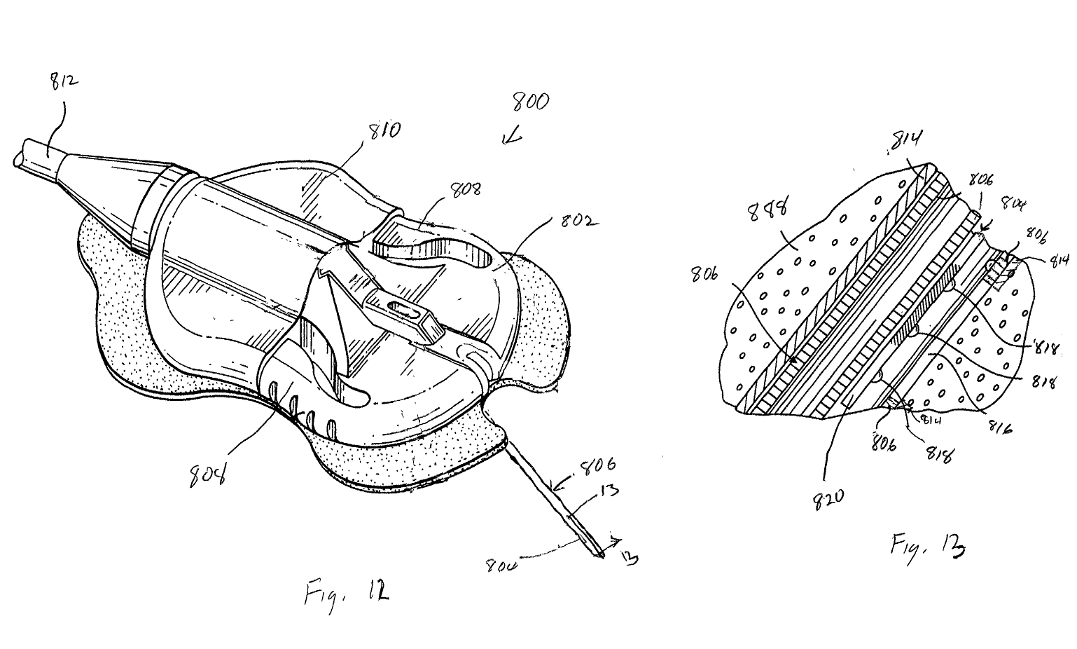

FIG. 1 illustrates an infusion set 10 in accordance with a first embodiment of the present invention. The infusion set 10 includes a base 12, a septum 14, a cannula housing 16 and a cannula 18. As shown in the illustrated embodiment, the infusion set 10 is adapted for a top disconnect of an infusion tube 20 that is coupled to an infusion device (not shown). Typical top disconnect systems include those described in U.S. Pat. No. 4,755,173 to Konopka et al. and U.S. Pat. No. 5,545,143 to Fischell, which are herein incorporated by reference, or the like. However, in alternative embodiments, such as shown in FIG. 2, an infusion set 100 uses side disconnect systems such as shown in U.S. Pat. No. 5,545,152 to Funderburk et al. or U.S. Pat. No. 5,545,143 to Fischell, which are herein incorporated by reference, or the like, or a system that includes a permanently connected infusion tube such as shown in U.S. Pat. No. 4,755,173 to Konopka et al., which is herein incorporated by reference, or the like.

Preferably, the base 12 is a soft flexible material that conforms to and moves with the skin of the user. For instance, pliable polyurethane or silicone rubber may be used. However, alternative embodiments may utilize other silicone based polymers, polyvinyl chloride, plastic, rubber, or the like. In particular embodiments, the undersurface of the base 12 is provided with an adhesive 22 for adhering the infusion set 10 to the skin of the user. In further embodiments, the adhesive 22 may include an anti-bacterial and/or healing promotion substance (such as dexamethasone, or the like) that reduces the risk of infection and speeds the healing process once the infusion set 10 is removed. In alternative embodiments, the adhesive 22 may be omitted or augmented with an adhesive over and/or under dressing to further secure the infusion set 10 to the skin of the user. Typical over or under adhesives include, but are not limited to, IV 3000 by Smith & Nephew, or the like.

In preferred embodiments, the infusion set 10 includes a self-sealing septum 14 that is secured to the cannula housing 16 and/or base 12 to provide a seal for the fluid path through the cannula 18 when an infusion tube 20 is connected to the infusion set 10. Preferably, the septum 14 is a pre-slit, separate septum such as disclosed in U.S. Pat. No. 4,755,173 to Konopka et al., which is herein incorporated by reference. However, alternative embodiments may utilize a non-slit septum, a valve, or the like. Further embodiments may utilize a septum formed integral with the infusion set, such as shown in U.S. Pat. No. 5,545,143 to Fischell, which is herein incorporated by reference. In addition, the infusion set 10 includes a cannula housing 16 that secures the cannula 18 (described in more detail below) to the infusion set 10. The cannula housing 16 may also provide an internal volume and structure for receiving fluid from the infusion tubing 20 and directing it into the skin of the user.

The cannula 18 of the infusion set 10 is generally formed from a flexible material, such as polyurethane, polyethylene, or the like. However, alternative embodiments may use other materials, such as PVC, plastic, micro-dialysis fiber, glass tubing, or the like. Preferably, the cannula 18 is formed in a manner and attached to the cannula housing as disclosed in U.S. Pat. No. 4,755,173 to Konopka et al. and U.S. Pat. No. 5,545,143 to Fischell, which are herein incorporated by reference, or the like. Preferably, the cannula has a diameter in the range equivalent to a 22 gauge to 30 gauge needle. Although other embodiments may utilize larger or smaller diameters.

Unlike typical infusion sets, the cannula 18 of the infusion set 10 in accordance with embodiments of the present invention is configured to permit insertion into the skin of the user 20 without the need of a sharp needle, or the like. The cannula 18 is reinforced and stiffened by a fluid soluble coating (or material) 24. In preferred embodiments, the coating 24 is on the exterior of the cannula 18. However, alternative embodiments may include the coating on the interior as well as (or instead of) the coating 24 on the exterior of the cannula 18. Preferably, the coating 24 also provides a sharp tip (or point) 26 that pierces the skin and guides the stiffened cannula 18 into the skin in a manner similar to that of a needle augmented infusion set. However, unlike a sharp needle, the coating 24 and sharp tip 26 dissolves in the bodily fluids of the user. Then the cannula 18 becomes flexible and dull so that there is minimal (or even a non-existent risk) of needle sticks upon removal of the infusion set 10 from the skin of the user. Thus, a user does not need to insert the infusion set 10 with a needle, withdrawal the needle and then dispose of it in an acceptable manner (such as a sharps container). Instead, the user simply inserts the infusion set 10 into the skin without an insertion needle and the soluble coating 24 dissolves over a period of time to leave a non-sharp, flexible cannula that is similar in comfort to the cannula of more traditional infusion sets. When the infusion set 10 is removed, the user can simply dispose of the infusion set 10 without concern for sticks from contaminated needles or sharps. In preferred embodiments, the coating 24 dissolves over time in the range of 5 to 20 minutes. However, shorter periods of time under a minute or longer periods of several hours (or in some cases days) may be used based upon user comfort, priming requirements, inclusion of additives, strength of the coating 24 and cannula 18, method of insertion, type of tissue inserted into, or the like.

As shown in FIG. 1, the soluble coating 24 is placed along the entire length of the exterior of the cannula 18. It may also contact the base and/or cannula housing 16 for improved structural strength and stability. Increasing the coating thickness at a point of stress, such as at the joint between the base 12 and the cannula 18, would also improve strength and stability. The coating 24 extends slightly beyond the end of the cannula 18 to produce a sharp tip 26 that is sufficiently sharp to allow substantially pain free insertion of the cannula 18 into the skin of the user. Preferred embodiments of the insertion set 10 are inserted into the skin of the patient utilizing an automatic insertion device, such as those disclosed in U.S. Pat. No. 6,093,172 to Funderburk et al. and PCT application publication No. WO 99/33504, which are herein incorporated by reference. The coating 24 thickness and shape of the tip 26 are selected depending on the diameter and thickness of the cannula 18, the material from which the cannula 18 is formed, the length of the cannula 18, the type of tissue the infusion set 10 is inserted into, the type of fluid to be infused, the desired time for the coating 24 to dissolve in the bodily fluids of the user, the speed of insertion, or the like. FIG. 3 illustrates a cross-section of the cannula 18 and coating 24 of the infusion set 10 as shown along the line 3-3 in FIG. 1. Generally, the coating 24 is thicker towards a base of the cannula 18 and gradually tapers to define the sharp tip 26. In preferred embodiments, the coating 24 is injection molded with a cannula 18 inserted into a mold prior to molding. However, in alternative embodiments, the coating 24 is applied by dipping, spraying, sintering, powder coating, precipitation from a supersaturated solution, poured molding, a combination of methods, or the like. In particular embodiments, the sharp tip 26 may be sharpened and shaped after the molding process, or the like. In alternative embodiments, the sharp tip 26 may be formed by cutting, spot melting, drawing, forming, abrasion, or the like, to produce bevels comparable to those of metal needles. A sufficiently sharp tip 26 reduces the pressure required for penetration of the skin and will reduce pain associated with insertion.

In the embodiment in FIG. 1, there is a bore 28 through a tip of the cannula 18 and coating to permit easy priming of the infusion set 10. However, like hollow needles in traditional infusion sets, the sharp tip 26 formed by the cannula 18 and coating 24 is still sufficiently sharp to permit easy and relatively pain free insertion into the skin of the user. To facilitate relatively pain-free insertion, the edges forming the tip 26 of the coating 24 are sharp enough to penetrate the skin much like a hollow needle that is inserted and used to inject fluids. In alternative embodiments, the bore 28 may be offset to provide at least one side of the sharp tip 26 with a thicker cross-section to provide greater structural stability.

Preferred embodiments of the present invention utilize sugar, or sugar-like materials, to form the coating 24 on the cannula 18. The sugar is heated and melted into a flowable material that can be applied by molding, dipping, spraying, sintering, or the like. The properties of the coating 24 are controlled by the selection of melting temperature, material that may be mixed in with the sugar or the like, the cooling rate of the sugar, the density of the coating 24, or the like. In addition, consideration is given to the amount of time required for the coating 24 to dissolve in the bodily fluids of the user. For instance, the coating 24 should not dissolve too quickly, because the sharp tip 26 of the coating 24 would dissolve during priming and prior to insertion of the infusion set 10. In addition, the coating 24 should not take too long to dissolve because, the longer the coating 24 remains in place, the greater the chance for discomfort increases or the greater the potential for sticks after removal of the infusion set 10.

Due to the nature of sugars, the candy making art provides guidance on the properties that can be achieved when forming the coating on the cannula. For instance, if sucrose is used as the underlying material, the material will have different properties based upon the temperature of the melted sucrose material, what it is mixed with, and how it is cooled. Sucrose mixed with water produces several different textures based upon specific temperature ranges. When heated to 270° to 290° F. (132° to 143° C.), the sucrose is in the “soft crack” phase and produces a coating that will bend and is not brittle. When heated to 300° to 310° F. (149° to 154° C.), the sucrose is in the “hard crack” phase and produces a coating that is hard and brittle. In particular embodiments, a coating of the “hard crack” material is applied. However, in alternative embodiments, multiple layers of “soft crack” and “hard crack” material are applied to provide a hard and sharp outer coating that is retained and strengthened by a more flexible layer to provide a composite structure more resistant to breakage.

Also, various combinations of sugars may be used, either in mixtures or as layers. In further embodiments, the cannula 18 and coating 24 could be engineered so that the sharp tip 26 is unlikely to be sharp enough for accidental sticks. For example, in this case, an unintentional stick is more likely to break the coating 24 and bend the cannula 18 to prevent the use of latter insertion. Other sugars that may be used include, but are not limited to, xylose, mannose, galactose, arabinose, glucose, xylitol, arabitol, sorbitol, galactitol, mannitol, monosaccharides, disaccharides, or the like. In alternative embodiments, a roughened surface is utilized to improve adhesion between the cannula 18 and/or coating layers. Still further embodiments may utilize fillers of different materials to enhance the structural properties of the coating 24. In other embodiments, one coating material is applied to the exterior of the cannula 18 and a different coating material is applied to the interior of the cannula to form a solid core.

Still further embodiments may utilize polysaccharides or the like. Some interesting polysaccharides are alginates, which are hard when dry, but soften when wet. But when the alginates dry again, they tend not to return to a sharp point. Another interesting polysaccharide is pectin. Due to the nature of polysaccharides, the material would be compression molded into the desired form when heated to a level that softens the material. Sintering may also be used. Melting should generally be avoided (unless under very controlled conditions), since this tends to cause the polysaccharide material to breakdown and/or bum. Other suitable materials include, but are not limited to, cyclodextrins (including α, β, and λ pyranose having an MP of 240 to 265). Cyclodextrins are used in controlled release applications and could be combined with sucrose, pectin, or other suitable materials to develop a composite that has desired properties.

Further embodiments may utilize other dissolvable materials, such as starches, polymers, artificial sugars, or the like. Typical water soluble polymers include, but are not limited to, polyvinyl alcohol, polyethylene oxide, polyethylene glycol, polyacrylamides, polyvinyl pyrolidone, polyacrylic acid, polycaplactone, polyorthoesters, or the like. These may be combined with other water soluble non-toxic, plasticizers, such as glycols, glycerols or the like to obtain desired hardness and dissolution times. Proteins such as gelatin, corn protein (i.e., Zein composed of amylose (approximately 27%) and amylopectin (approximately 73%)), or the like may be used. Starches include soluble starch (such as amylodextrin or the like) or insoluble starch (such as amylase or the like).

The choice of the material would depend on the structural strength required, the time period desired for the coating to dissolve, resistance of the coating to the fluid expelled during priming, speed of insertion, and location on the body where the insertion will occur. Other considerations may include sterilization methods, hydration rate of the coating material, inclusion of a desiccant in the packaging, the temperature and humidity typically encountered by the infusion set during storage, transportation and use. Further embodiments may include temperature and humidity stickers (that are included in the packaging), which may change color or appearance to indicate when the coating material has been compromised by temperature and/or humidity that would effect the usability of the insertion set. Further embodiments may include a color change material within the coating material to indicate excessive humidity and/or temperature.

Alternative embodiments of the coating materials may include additives, such as anti-microbial materials, anti-inflammatory materials, or the like. The concentrations may range from 0.1% to 3%, although larger or smaller concentrations may be used based on the properties of the additives and the coating materials selected for use with the insertion sets. In further alternative embodiments, the additives may be included in the coating materials by micro-encapsulation. In other embodiments, the coating material may be covered with a layer of lubricating material (such as silicone, glycerin, or the like) to facilitate insertion by making the coating and cannula more slippery. The covering layer may include a preservative, an anti-inflammatory, an antimicrobial, a moisture resistant barrier, or the like, the covering layer may be applied by spraying, dipping, brushing, baking, or the like.

FIGS. 4 and 5 illustrate cross-sectional views of a cannula 200 of an insertion set in accordance with a third embodiment of the present invention. This embodiment includes a coating 202 that uses side flanges (or rails) 204 to produce a generally star shaped cross-section. Each of the flanges 204 tapers out to an edge 206 and then down to a sharp tip 208. The use of flanges 204 provides greater structural support and rigidity for loads experienced during insertion of the infusion set to minimize breakage of the coating 202 or bending of the cannula 200. In alternative embodiments, the cannula may also have a matching cross-section to provide additional support to the side flanges. More or less flanges may be used depending on structural strength and patient comfort. Alternative embodiments may utilize other cross-sections.

FIG. 6 is a cross-section of a cannula 300 for use with an insertion set in accordance with a fourth embodiment of the present invention. A coating 302 extends along the surface of the cannula 300 and closes off a tip 304 of the cannula 300 with a solid sharp tip 306. If priming is desired, side ports 308 are placed in the side of the cannula 300 and coating 302. Then when the user primes the infusion set, the fluid emerges out of the side ports 308 and tends to avoid the sharp tip 306, which could result in premature dulling of the sharp tip 306 of the coating 302. In alternative embodiments, the fluid moves out of the side ports 308 and down towards the sharp tip 306 and partially dissolves the coating 302 near the side ports 308. This fluid then becomes saturated to provide a lubricating effect at or near the sharp tip 306 of the coating 302 when entering the skin of the user. Generally, in this case, the dulling of the sharp tip 306 is minimized, since the fluid from the side ports 308 is somewhat saturated prior to contacting the sharp tip 306. In alternative embodiments, the sharp tip 306 is protected within an outer coating of silicone, petroleum jelly, KY jelly, harder less soluble sugar, or the like, to postpone the dissolving of the sharp point by the fluid from the side ports.

FIG. 7 is a cross-section of a cannula 400 for use with an insertion set in accordance with a fifth embodiment of the present invention. The cannula 400 in this embodiment is formed from a porous material. This provides several advantages. For instance, the porous nature of the cannula 400 will provide for better adhesion of a coating 402 to provide greater structural support during insertion. Thus, the structure more closely resembles a composite rather than a laminate. In addition, the porous nature may speed dissolving of the coating 402, once the infusion set is inserted into the skin of the user and the cannula 400 is filled with fluid. In particular embodiments, the cannula 400 is only porous for a portion of its length, preferably, towards the tip 404 of the cannula 400, so that the fluid does not leave the cannula 400 too close to the surface of the skin of the user. Preferably, in this embodiment, the infusion set would be primed up to the base (not shown) of the set to avoid prematurely dissolving the material 402. After insertion a small priming bolus would be delivered to fill up the empty space and to assist in dissolving the material 402.

FIG. 11 illustrates an alternative embodiment that includes a sealed porous cannula 400′ with a closed end 420 that is also porous. The cannula 400′ is coated with material 402′ that has a sharp tip 422 that is supported by the closed end 420 to minimize the mechanical stresses on the sharp tip 422. Preferably, as described above, the infusion set would be primed up to the base (not shown) of the set to avoid prematurely dissolving the material 402′ and the sharp tip 422. After insertion a small priming bolus would be delivered to fill up the empty space and to assist in dissolving the material 402′ and the sharp tip 422.

FIG. 8 is a cross-section of a cannula 500 for use with an insertion set in accordance with a sixth embodiment of the present invention. In this embodiment, a coating 502 of the cannula 500 is provided internally to produce a solid core of material that ends in a sharp tip 504. The cannula 500 provides a support structure similar to a straw (or reinforcing casing) to keep the rigid coating 502 material from shearing off during insertion of the infusion set into the skin of the user. Generally, this infusion set would not be primed prior to insertion. After insertion, the center core of the coating 502 is designed to slide forward slightly upon the application of fluid to the end of the cannula 500. The coating 502 then dissolves as fluid passes over the core on its way into the skin of the user. In alternative embodiments, the cannula 500 stretches slightly under the pressure from the fluid to provide a fluid passage around the dissolving coating 502. In this embodiment, it is preferred to have a relatively quickly dissolving coating 502 that takes only a few minutes to soften and provide a fluid path. This type of cannula would be best suited for an infusion set with an “at site” disconnect system, where the infusion set tubing could be primed prior to attachment to the infusion set. In another embodiment, the solid core may be formed with a longitudinal groove to allow fluid to flow along the groove and the interior of the cannula and out the tip.

FIG. 9 is a cross-section of an infusion set 600 with a cannula 602 in accordance with a seventh embodiment of the present invention. In this embodiment, the cannula 602 is provided with a coating 604 that provides a sharp tip 606 for insertion into the skin of the user. However, the cannula 602 also includes an internal step 608 to receive and stop a solid wire (or a tube, blunt plastic rod, or the like) insert 610 that serves to stiffen and support the cannula 602 and coating 604 during insertion of the infusion set 600 into the skin of the user. After insertion, the insert 610 is withdrawn and the infusion tubing (not shown) is connected. Due to the protective coating 604, the insert 610 is unlikely to be contaminated with bodily fluids. Since the insert 610 is not sharp (and may not be contaminated), disposal of the insert 610 is simplified and the possibility of an accidental stick (or contamination) is substantially reduced (or eliminated).

FIG. 10 is a cross-section of an infusion set 700 that utilizes a flexible metal cannula 702 in accordance with an eighth embodiment of the present invention. In this embodiment, the cannula 702 is a metal needle that does not include a sharp tip. Rather a coating forming a sharp tip 704 of soluble material is provided at the end of the metal cannula 702 to provide a tip that is sharp enough to facilitate insertion. Thus, after insertion, the coating forming the sharp tip 704 dissolves to leave a needle that is no longer sharp so that the chance of inadvertent sticks is substantially reduced (or eliminated). In alternative embodiments, the cannula 702 may be made out of other materials, such as ceramic, hard plastics, non-flexible metal, or the like. Also, the cannula 702 may be coated with soluble material in addition to the sharp tip 704.

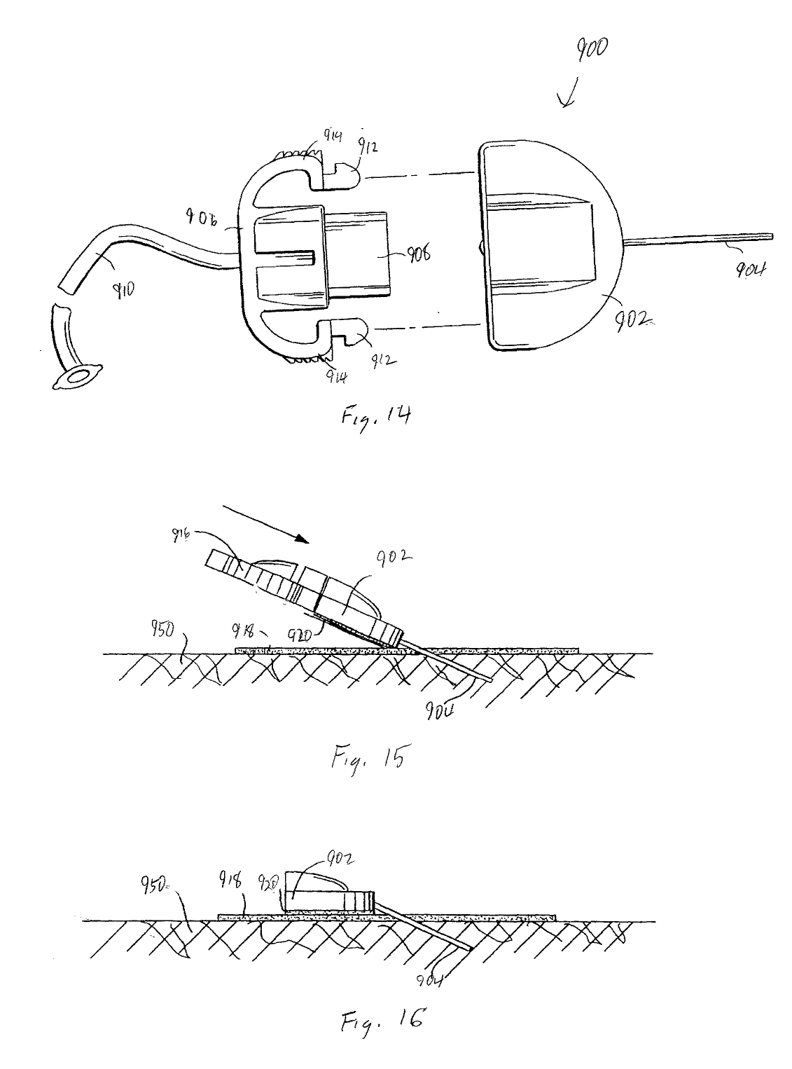

FIGS. 12 and 13 illustrate a sensor set 800 in accordance with a ninth embodiment of the present invention. The sensor set 800 includes a base 802, a sensor 804, a cannula 806 and a connector 808 for connection to a cable connector 810 and cable 812. In alternative embodiments, the sensor set 800 may connect to a telemetered transmitter or other device, such as disclosed in U.S. patent application Ser. No. 09/377,472 filed Aug. 19, 1999 (or PCT application publication No. WO 00/19887), which is herein incorporated by reference. In particular embodiments, a coating 814 is applied over the cannula 806, except for a window area 816 over electrodes 818 of the sensor 804 to expose the sensor to the bodily fluids of the user present in the skin (or other tissues of the user) 888. In alternative embodiments, the coating 814 is applied to the entire sensor 804 and the electrodes 818. However, the electrodes 818 may require an additional coating or membrane to prevent the material forming the coating 814 from interacting or affecting the chemistry attached to the electrodes 818 during storage or during dissolution of the coating 814. In further alternative embodiments, the cannula 806 may be omitted and the coating 814 may be applied directly to the sensor 804 and the substrate 820 that supports the electrodes 818 of the sensor 804. After insertion, the soluble coating material, dissolves. Sensors may be flexible or non-flexible. Typical sensors that can be used with this embodiment include, but are not limited to, U.S. Pat. No. 5,391,250 issued Feb. 21, 1995 to Cheney II, et al.; U.S. Pat. No. 5,390,671 issued Feb. 21, 1995 to Lord et al.; U.S. Pat. No. 5,954,643 issued Sep. 21, 1999 to Van Antwerp et al.; U.S. Pat. No. 5,108,819 issued Apr. 28, 1992 to Heller et al.; and U.S. Pat. No. 6,103,033 issued Aug. 15, 2000 to Say et al., all of which are herein incorporated by reference.

One possible additional advantage to coating a glucose sensor with a sugar or saccharide coating is that the coating may dissolve in a predictable manner. If the coating dissolves predictably, the readings obtained during the dissolving of the coating can be used to calibrate the sensor using a known decay curve. For instance, embodiments could use the rate or slope as opposed to a change in signal magnitude. In alternative embodiments, a covering or coating layer that delays the dissolving of the soluble coating material until after the sensor is stabilized in the body (typically, 10 minutes to several hours), after which the coating quickly dissolves to create a detectable signal. Some embodiments might require multiple layers.

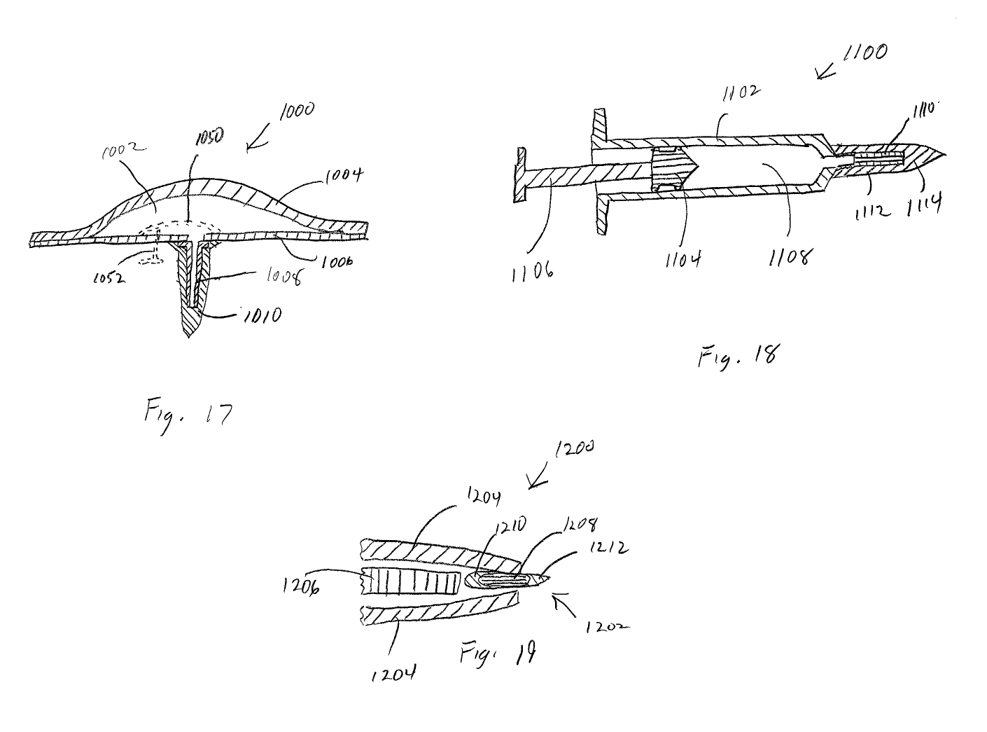

FIGS. 14-16 illustrate an angled infusion set 900 in accordance with a tenth embodiment of the present invention. The infusion set 900 includes a main body 902 that supports a cannula 904 (with a soluble coating of material as described above) for insertion into the skin 950 of a user. The infusion set 900 utilizes an at-site disconnect that includes a connector body 906 and set connector 908 for connecting infusion tubing 910 to the main body 902 of the infusion set 900. The connector body 906 includes finger grips 914 and lock tabs 912 to secure the connector body 906 to the main body 902. The insertion set 900 is adapted for placement of an angled infusion cannula 904, as opposed to a 90° cannula as discussed above, since some users prefer an angled infusion set for comfort and/or profile. To insert the infusion set 900, the user attaches an insertion body 916 to the main body 902 to make the infusion set 900 easier to manipulate and inserts it at an angle on the skin. Preferably, the cannula 904 is inserted through tape 918. Although other embodiments may include a hole in tape 918 or omit tape 918 and use an over dressing. After insertion of the cannula 904, the insertion body 916 is removed and the main body is pressed flat against the tape 918. Preferably, the main body 902 includes an adhesive 920 to firmly secure the main body 902 to the tape 918. Finally, the connector body 906 is connected to the main body 902 to provide fluid connection between the infusion tubing 910 and the cannula 904. After insertion, the soluble coating of material dissolves.

FIG. 17 illustrates an infusion device 1000 in accordance with an eleventh embodiment of the present invention. The infusion device 1000 includes fluid 1002 that is under pressure by a flexible top surface 1004 and a rigid bottom surface 1006. In alternative embodiments, other methods, such as gas pressurization, spring compression of two rigid walls, moveable pistons, or the like, may be used to maintain the required pressure to deliver the fluid 1002. The infusion device 1000 also includes a cannula 1008 (with a soluble coating of material 1010 as described above) for insertion into the skin of a user. The cannula 1008 is blocked by the coating material 1010 to prevent delivery of the fluid until after insertion of the cannula 1008 and the coating material 1010 has dissolved. In preferred embodiments, the infusion device 1000 is filled with fluid 1002 just prior to use. In alternative embodiments, if the coating material 1010 is insoluble in the fluid 1002, the infusion device 1000 may be pre-filled at the factory. In further alternatives, a valve is opened or a slide cover is removed from the cannula 1008 of a pre-filled infusion device 1000, just prior to or immediately following insertion. In further alternative embodiments, a septum (or diaphragm) 1050 is pierced by pin 1052 (shown in dashed lines in FIG. 17) when an infusion device is placed on the skin. Preferably, the coating material 1010 on the cannula 1008 is a material that dissolves in the bodily fluids predictably over time, so that the fluid 1002 may be delivered at a desired time after insertion of the infusion device 1000. For example, if growth hormone is desired to be delivered, it is best delivered to the user between 2 to 4 a.m., and the coating material 1010 may be selected to dissolve between 6 to 8 hours after being placed on the user. Thus, a user can place the infusion device 1002 and insert the cannula 1008 just prior to going to sleep so that the growth hormone is delivered at the desired time. The size of the cannula 1008 and the fluid pressure can be selected to deliver the fluid over several minutes or several hours depending on the type of fluid and the desired infusion rate. Typical dissolve times can range from minutes to several hours and volumes would be dependent on the fluid to be delivered. After use, the user can remove and discard the infusion device 1000 without concerns of accidental sticks from the cannula 1008.

FIG. 18 illustrates a syringe device 1100 in accordance with a twelfth embodiment of present invention. The syringe device 1100 includes a syringe body 1102 that mates with a piston 1104 that is moved within the syringe body 1102 by a plunger 1106 to expel fluid 1108 contained within the syringe device 1100. Instead of using a traditional rigid needle, the syringe device 1100 utilizes a cannula 1110 that includes a soluble coating of material 1112 to provide a sharp tip 1114 to facilitate insertion of the syringe device 1100 and administration of an injection. In preferred embodiments, the cannula 1110 and the coating material are assembled with the syringe body 1102 at the time of manufacture. However, in alternative embodiments, the cannula 1110 and coating material 1112 are formed as a separate piece that may be attached just prior to an injection. In addition, if provided with a standard Luer connector at the base of the cannula 1110, the cannula 1110 and the coating material 1112 may be used to adapt a standard syringe for needle-less operation. In further alternative embodiments, the syringe device may be other devices, such as pen-type injectors (some of which utilize pre-filled cartridges or reservoirs), syringe infusion devices, fluid transfer devices, or the like.

After insertion of the cannula 1110 into the skin, the sharp tip 1114 of the coating material 1112 is designed to slide forward slightly (or break off) upon the application of fluid to the end of the cannula 1110 to allow the fluid 1108 to escape during the injection. The coating material 1112 dissolves as fluid passes over it on the way into the skin of the user, and the coating material 1112 may completely dissolve during the injection. Alternatively, the coating material 1112 breaks off in the skin during removal, after which it then dissolves over a period of time in the skin. In alternative embodiments, the cannula 1110 stretches slightly under the pressure from the fluid to provide a fluid passage around and/or through the dissolving coating material 1112 and the sharp tip 1114 remains in the skin as it dissolves. In alternative embodiments, the coating material may be formed with small barbs, hooks, or the like to facilitate retention of the sharp tip 1114 in the skin so that it is not withdrawn with the cannula 1110 after an injection.

FIG. 19 illustrates an insertion device 1200 and element 1202 in accordance with a 25 thirteenth embodiment of the present invention. The insertion device 1200 includes a housing 1204 and a plunger 1206. The housing 1204 holds the element 1202 and the plunger 1206. The element includes a core 1208 to be implanted in the skin of the user and a soluble coating material 1210 with a sharp tip 1212 to facilitate insertion. The housing 1204 is placed against the skin and the plunger 1206 is activated to thrust the element 1202 through the skin. After insertion, the housing 1204 is removed and the element 1202 remains in the skin where the coating material 1210 and the sharp tip 1212 dissolve to leave the core 1208. In preferred embodiments, the plunger 1206 stops at the surface of the skin. However, in alternative embodiments, the coating material 1210 extends a distance behind the core 1208 and the plunger 1206 does not contact the skin or bodily fluids during insertion. Rather the extended end of the coating material 1210 breaks off and/or dissolves away over a period of time. This embodiment is suitable for placing Norplant®, pellet medications, implantable sensors, implantable devices, osmotic pumps, or the like.

It is noted that embodiments of the present invention are directed to insertion sets and that the various illustrated embodiments may be used and combined in different manners and may be utilized with infusion sets, sensor sets and/or infusion devices. Further embodiments may be utilized with other devices that are used to pierce the skin or tissue of a user.

While the description above refers to particular embodiments of the present invention, it will be understood that many modifications may be made without departing from the spirit thereof. The accompanying claims are intended to cover such modifications as would fall within the true scope and spirit of the present invention.

The presently disclosed embodiments are therefore to be considered in all respects as illustrative and not restrictive, the scope of the invention being indicated by the appended claims, rather than the foregoing description, and all changes which come within the meaning and range of equivalency of the claims are therefore intended to be embraced therein.

What is claimed is:

1. An insertion set for insertion into a skin of a user, the insertion set comprising:
an insertable substantially insoluble flexible portion capable of remaining in the skin after insertion; and
a soluble material coupled to the insertable substantially insoluble flexible portion that facilitates piercing the skin, and wherein the soluble material dissolves in the skin of the user.

2. The insertion set according to claim 1, wherein the soluble material holds the insertable substantially insoluble portion in a rigid state.

3. The insertion set according to claim 1, wherein the insertable substantially insoluble flexible portion is a cannula.

4. The insertion set according to claim 3, wherein the cannula is formed from a flexible plastic material.

5. The insertion set according to claim 1, wherein the insertion set is an infusion set.

6. The insertion set according to claim 1, wherein the soluble material is formed from at least one saccharide.

7. The insertion set according to claim 1, wherein the soluble material is formed from at least one starch.

8. The insertion set according to claim 1, wherein the soluble material is formed from at least one protein.

9. The insertion set according to claim 1, wherein the soluble material dissolves in the skin in under ten minutes.

10. The insertion set according to claim 1, wherein the soluble material dissolves in the skin in under 1 hour.

11. The insertion set according to claim 1, wherein the soluble material dissolves in the skin in over 1 hour.

12. The insertion set according to claim 1, wherein the insertion set is adapted to be inserted into subcutaneous tissue.

13. The insertion set according to claim 1, wherein the soluble material includes at least one flange to improve structural strength.

14. The insertion set according to claim 1, wherein the soluble material is formed from multiple layers of materials with different properties.

15. The insertion set according to claim 1, wherein the insertable substantially insoluble flexible portion is a cannula with at least one side port.

16. The insertion set according to claim 1, wherein the insertion set is an infusion set that includes an at site disconnect for use with infusion tubing.

17. The insertion set according to claim 1, wherein the insertion set is an infusion set that includes a side disconnect for use with infusion tubing.

18. The insertion set according to claim 1, wherein the insertable substantially insoluble flexible portion is a sensor.

19. The insertion set according to claim 18, wherein the insertable substantially insoluble flexible portion further includes a cannula surrounding the sensor.

20. The insertion set according to claim 1, wherein the insertable substantially insoluble flexible portion is porous.

21. An infusion device for insertion into a skin of a user to infuse a fluid, the infusion device comprising:
an insertable substantially insoluble flexible portion capable of remaining in the skin after insertion to deliver a fluid; and
a soluble material coupled to the insertable substantially insoluble flexible portion that facilitates piercing the skin, and wherein the soluble material dissolves in the skin of the user.

22. A syringe device for insertion into a skin of a user to deliver an injection, the syringe device comprising:
an insertable substantially insoluble flexible portion capable of delivering an injection to the skin of the user after insertion; and
a soluble material coupled to the insertable substantially insoluble flexible portion that facilitates piercing the skin, and wherein the soluble material dissolves in the skin of the user.

23. An element for insertion into a body of a user, the insertion element comprising:
an insertable substantially insoluble portion capable of remaining completely within the body after insertion; and
a soluble material coupled to the insertable substantially insoluble portion that facilitates piercing the body, and wherein the soluble material dissolves in the body of the user to leave the insertable substantially insoluble portion completely within the body.

24. A sensor set for insertion into a body of a user, the sensor set comprising:
an insertable substantially insoluble portion capable of remaining in the body after insertion; and
a soluble material coupled to the insertable substantially insoluble portion that facilitates piercing the body, and wherein the soluble material dissolves in the body of the user.

25. The sensor set according to claim 24, wherein the insertable substantially insoluble portion includes a cannula.

26. The sensor set according to claim 25, wherein the cannula is formed from a flexible material.

27. The sensor set according to claim 25, wherein the cannula is a flexible metal tube.

28. The sensor set according to claim 24, wherein the soluble material is formed from at least one saccharide.

29. The sensor set according to claim 24, wherein the soluble material is formed from at least one starch.

30. The sensor set according to claim 24, wherein the soluble material is formed from at least one protein.

31. The sensor set according to claim 24, wherein the soluble material dissolves in the body in under ten minutes.

32. The sensor set according to claim 24, wherein the soluble material dissolves in the body in under 1 hour.

33. The sensor set according to claim 24, wherein the soluble material dissolves in the body after 1 hour.

34. The sensor set according to claim 24, wherein the soluble material is comprised of at least two coatings, wherein one of the at least two coatings dissolves slowly and another of the at least two coating dissolves quickly.

35. The sensor set according to claim 24, wherein the sensor set is adapted to be inserted through the skin of the body.

36. The sensor set according to claim 24, wherein the sensor set is adapted to be inserted into subcutaneous tissue.

37. The sensor set according to claim 24, wherein the soluble material includes at least one flange to improve structural strength.

38. The sensor set according to claim 34, wherein the soluble material is formed from multiple layers of materials with different properties.

39. The sensor set according to claim 24, wherein the insertable substantially insoluble portion is a cannula with at least one side port.

40. The sensor set according to claim 24, wherein the insertable substantially insoluble portion is a sensor.

41. The sensor set according to claim 40, wherein the insertable substantially insoluble portion further includes a cannula surrounding the sensor.

42. The insertion set according to claim 24, wherein the insertable substantially insoluble portion is porous.

43. An infusion set for insertion into a skin of a user to infuse a fluid, the infusion set comprising:
an insertable substantially insoluble rigid portion capable of remaining in the skin after insertion; and
a soluble material coupled to the insertable substantially insoluble rigid portion that facilitates piercing the skin, and wherein the soluble material dissolves in the skin of the user.
